(19) United States
(12) Patent Application Publication
(10) Pub. No.: US 2025/0213780 A1
(43) Pub. Date: Jul. 3, 2025

(54) Cord Sling Device

(71) Applicant: Adam Lutz, Zelienople, PA (US)

(72) Inventor: Adam Lutz, Zelienople, PA (US)

(21) Appl. No.: 18/954,759

(22) Filed: Nov. 21, 2024

Related U.S. Application Data
(60) Provisional application No. 63/615,544, filed on Dec. 28, 2023.

(51) Int. Cl. A61M 5/14

(52) U.S. Cl. CPC A61M 5/1418

(57) ABSTRACT

A cord sling device is described and comprises an organizer used to hold a multitude of cords on medical devices such as IV poles. The cord sling device comprises a body component with adjustable fasteners designed to hang up the device and tie up cords and cables. Thus, once the device is hung from an IV pole or other equipment, and the wrapped cords are hung from the adjustable cord fasteners, a user can ensure a safer, more organized, and visually appealing environment for cords and cables. Further, the cord sling device protects cords and cables from wear and tear, or from becoming separated and unprotected thereby shortening the life span of the cords and cables. Also, the cord sling device enables for better airflow and prevents possible overheating issues with electronic devices.

### CROSS-REFERENCE TO RELATED APPLICATION

The present application claims priority to, and the benefit of, U.S. Provisional Application No. 63/615,544, which was filed on Dec. 28, 2023, and is incorporated herein by reference in its entirety.

### FIELD OF THE INVENTION

The present invention relates generally to the field of cord sling devices. More specifically, the present invention relates to a sling to hold the cords of an IV pump while it is held on an IV pole. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in cord sling devices. Generally, unruly cords on an IV pole can be dangerous to both a healthcare worker and patient. A patient may try to get up and use the restroom and accidentally trip on long unorganized cords and cause injury and pain. It is also a tripping hazard for healthcare professionals.

As is known, multiple cords hanging from an IV pole can be unsightly and often become tangled. Multiple tangled cords create an unsafe and cluttered environment, making already difficult circumstances worse. Additionally, cords can suffer wear and tear over time if they are tangled on the floor, potentially becoming separated and unprotected. Further, having multiple cords on top of each other can cause overheating issues, thereby damaging expensive electronic devices.

Accordingly, there is a demand for an improved cord sling device that neatly holds cords up in an organized fashion.

Therefore, there exists a long felt need in the art for a cord sling device that provides users with a safer, more organized, and visually appealing environment for cords and cables. There is also a long felt need in the art for a cord sling device that reduces clutter and eliminates tangled cords and cables on a job site, in hospitals, or in home environments. Further, there is a long felt need in the art for a cord sling device that protects cords and cables from wear and tear, or from becoming separated and unprotected, shortening the life span of the cords and cables. Moreover, there is a long felt need in the art for a device that enables for better airflow and prevents possible overheating issues with electronic devices. Finally, there is a long felt need in the art for a cord sling device that can be cleaned between patients and reused if desired.

The subject matter disclosed and claimed herein, in one embodiment thereof, comprises a cord sling device. The device is a cord sling that neatly holds cords up in an organized fashion. The cord sling device provides users with a safer, more organized, and visually appealing environment for cords and cables. Additionally, the cord sling device reduces clutter and eliminates tangled cords and cables on a job site, in hospitals, or in home environments. Further, the cord sling device protects cords and cables from wear and tear, or from becoming separated and unprotected thereby shortening the life span of the cords and cables. Also, the cord sling device enables for better airflow and prevents possible overheating issues with electronic devices. Finally, the cord sling device can be cleaned between patients and reused if desired.

In this manner, the cord sling device of the present invention accomplishes all of the forgoing objectives and provides users with a device that enables for a safer, more organized, and visually appealing environment for cords and cables.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a cord sling device. The device is an organizer used to hold a multitude of cords on medical devices such as IV poles. The cord sling device comprises a body component with adjustable fasteners designed to hang up the device and tie up cords and cables. Thus, once the device is hung from an IV pole or other equipment, and the wrapped cords are hung from the adjustable cord fasteners, a user can ensure a safer, more organized, and visually appealing environment for cords and cables.

In one embodiment, the cord sling device of the present invention assists any setting in which electronic devices comprising cords and cables are used. The cord sling device secures easily to an IV pole or other equipment and retains a plurality of cords and cables in a neat and organized fashion. The device is portable in that it is configured to be easily carried by a person.

In one embodiment, the cord sling device comprises a body component. The body component is typically configured in a rectangular (i.e., rectilinear) shape but can be configured in any suitable shape as is known in the art. Typically, the body component comprises holes, slots, a top loop fastener, and cord fasteners.

In one embodiment, the body component comprises one or more holes towards the top of the device. The body component also comprises a top loop fastener that is generally T-shaped and flexible enough to fit into one of these holes and sturdy enough to support the weight of the device and cords and cables suspended therefrom once the cords and cables are secured thereto.

In one embodiment, the body component of the cord sling device comprises cord fasteners and slots. The cord fasteners are generally rectangular (i.e., rectilinear) in shape but comprise a plurality of ridges along both of its longer sides. In use, the user inserts the cord fasteners into the slots, encompassing wrapped cords and cables. The ridges help secure the cord fasteners within the slots and provide for an adjustable attachment depending on the number, weight, and width of various cords and cables. In the present embodiment, the ridges on the cord fasteners act as a zip-tie-like fastener, although the cords and cables may be secured to the device via a buckle, snap, hook and loop fastener, etc., or any other suitable securing means as is known in the art.

In one embodiment, the cord sling device in accordance with the present invention can be produced in various colors, designs, patterns, etc., and feature logos, emblems and or designs, such as a medical facility requests and/or a user desires.

In one embodiment, the cord sling device is manufactured of a high-density polyethylene (HDPE), or any other suitable materials as is known in the art. Any number of different types of materials can be used to make the cord sling device including but not limited to plastics, nylon, etc.

In use, the user secures the device to an IV pole or other equipment pole by inserting the top loop fastener into the desired hole found on the top portion of the body component. A user then wraps the cords and cables in a neatly spooled fashion. Next, a user can secure the cords and cables to the device by inserting the cord fasteners into the slots found on the body component. The user can then utilize the device to retain a plurality of cords and cables. Thus, the cord sling device provides users with a safer, more organized, and visually appealing environment for cords and cables. Further, the device protects cords and cables from wear and tear and protects medical equipment from overheating issues by providing better airflow. The device can also be easily cleaned between patients and reused if desired.

It will also be appreciated that there are a number of additional add-on features that can be incorporated into the device and moreover, the cord sling device can take many different forms as is known in the art.

In yet another embodiment, the cord sling device comprises a plurality of indicia.

In yet another embodiment, a method of conveniently retaining a plurality of cords and cables. The method includes the steps of providing a cord sling device comprising a body component, a top loop fastener, and cord fasteners. The method also comprises inserting the top loop fastener into the holes at the top of the body component. Further, the method comprises hanging the device from an IV pole or other medical equipment via the top loop fastener. Next, the method comprises wrapping up cords in a neatly spooled fashion. Additionally, the method comprises inserting the desired cord fasteners into their respective slots. Finally, the method comprises utilizing the device to retain a plurality of cords and cables.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the cord sling device of the present invention showing two different lengths of the device in accordance with the disclosed architecture;

FIG. 2 illustrates a perspective view of one embodiment of the cord sling device of the present invention showing how the first version is secured to support one or more cords in accordance with the disclosed architecture;

FIG. 3 illustrates a perspective view of one embodiment of the cord sling device of the present invention showing how the second version is secured to support one or more cables in accordance with the disclosed architecture;

FIG. 4 illustrates a perspective view of one embodiment of the cord sling device of the present invention showing the device in use on a medical device and how it supports cords and cables in accordance with the disclosed architecture;

FIG. 5 illustrates a perspective view of one embodiment of the cord sling device of the present invention showing the second version of the organizer in use to support more than one set of cords; and

FIG. 6 illustrates a flowchart showing the method of conveniently retaining a plurality of cords and cables in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a cord sling device that provides users with a safer, more organized, and visually appealing environment for cords and cables. There is also a long felt need in the art for a cord sling device that reduces clutter and eliminates tangled cords and cables on a job site, in hospitals, or in home environments. Further, there is a long felt need in the art for a cord sling device that protects cords and cables from wear and tear, or from becoming separated and unprotected, shortening the life span of the cords and cables. Moreover, there is a long felt need in the art for a device that enables for better airflow and prevents possible overheating issues with electronic devices. Finally, there is a long felt need in the art for a cord sling device that can be cleaned between patients and reused if desired.

The present invention, in one exemplary embodiment, is a novel cord sling device for neatly hanging up cables and cords of various electronic medical equipment and other devices. The device is an organizer used to hold a multitude of cords on medical devices such as IV poles. The cord sling device comprises a body component with adjustable fasteners designed to hang up the device and tie up cords and cables. Thus, once the device is hung from an IV pole or other equipment, and the wrapped cords are hung from the adjustable cord fasteners, a user can ensure a safer, more organized, and visually appealing environment for cords and cables. The present invention also includes a novel method of conveniently retaining a plurality of cords and cables. The method includes the steps of providing a cord sling device comprising a body component a top loop fastener, and cord fasteners. The method also comprises inserting the top loop fastener into the holes at the top of the body component. Further, the method comprises hanging the device from an IV pole or other medical equipment via the top loop fastener. Next, the method comprises wrapping up cords in a neatly spooled fashion. Additionally, the method comprises inserting the desired cord fasteners into their respective slots. Finally, the method comprises utilizing the device to retain a plurality of cords and cables.

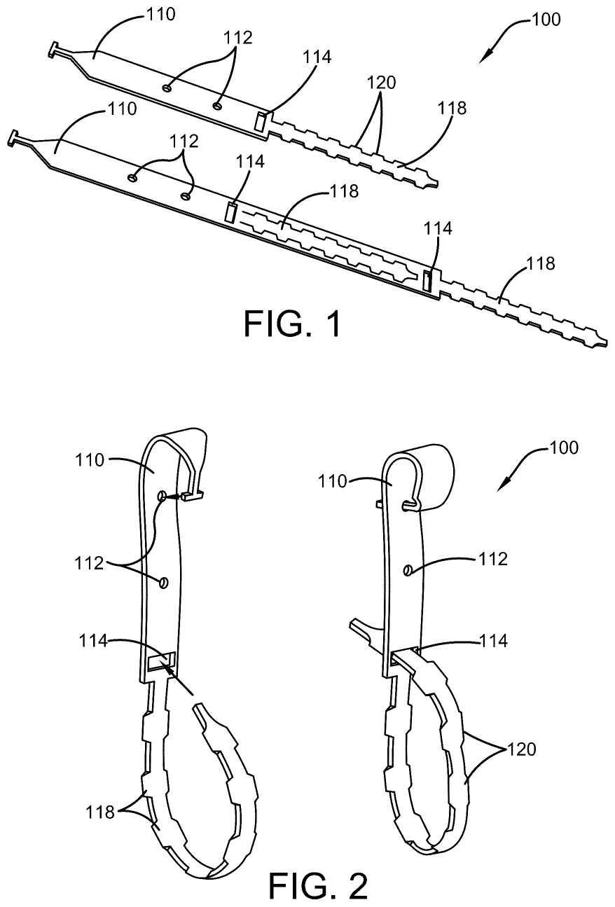

Referring initially to the drawings, FIG. 1 illustrates a perspective view of one embodiment of the cord sling device 100 of the present invention, showing two different lengths of the device 100. In the present embodiment, the cord sling device 100 is an improved cord sling device 100 that provides a user with a means of retaining a plurality of cords and cables 350 in a neat and organized fashion. Specifically, the cord sling device 100 comprises a body component 110 with holes 112, slots 114, a top loop fastener 116, and cord fasteners 118. Thus, once the device 100 is secured to an IV pole 300 or other equipment via inserting the top loop fastener 116 into the desired hole 112, and the cords and cables 350 are secured to the device 100 via inserting the cord fasteners 118 into the desired slots 114, the user can utilize the device 100 to retain a plurality of cords and cables 350 in a safer, more organized, and more visually appealing fashion.

The body component comprises a top loop fastener 116 male end that is generally T-shaped and flexible enough to fit into one of the holes 112 found on the body component 110 and sturdy enough to support the weight of the device 100 and cords and cables 350 once secured inside a hole 112.

Generally, the cord sling device 100 of the present invention assists any setting in which electronic devices comprising cords and cables 350 are used. The cord sling device 100 secures easily to an IV pole 300 or other equipment and retains a plurality of cords and cables 350 in a neat and organized fashion. The device is portable in that it is configured to be easily carried by a person.

As shown in FIG. 2, the cord sling device 100 comprises a body component 110. The body component 110 is typically configured in a rectangular (i.e., rectilinear) shape but can be configured in any suitable shape as is known in the art. Typically, the body component 110 comprises holes 112, slots 114, a top loop fastener 116, and cord fasteners 118. In FIG. 2, the insertions of the top loop fastener 116 male end into a hole 112 and a cord fastener 118 male end into a slot 114 are shown.

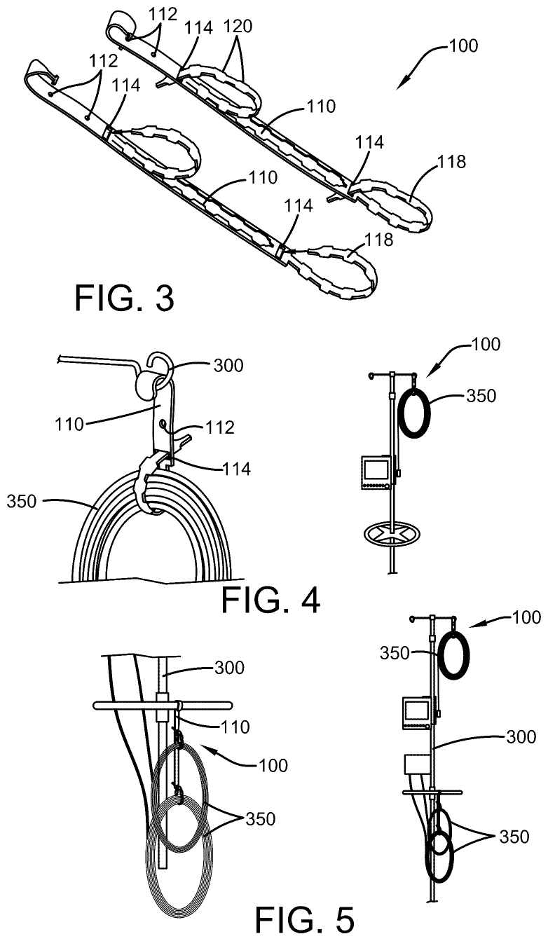

Shown in FIGS. 3 and 4, and in use, the user secures the device 100 to an IV pole 300 or other equipment by inserting the top loop fastener 116 into the desired hole 112 found on the top portion of the body component 110. A user then wraps the cords and cables 350 in a neatly spooled fashion. Next, a user can secure the cords and cables 350 to the device 100 by inserting the cord fasteners 118 into the slots 114 found on the body component 110. The user can then utilize the device 100 to retain a plurality of cords and cables 350. The cord fasteners 118 are generally rectangular (i.e., rectilinear) in shape but comprise a plurality of ridges 120 along both of their longer sides. In the present embodiment, the ridges 120 on the cord fasteners 118 act as a zip-tie-like fastener, although the cords and cables 350 may be secured to the device 100 via a buckle, snap, hook and loop fastener, etc., or any other suitable securing means as is known in the art.

Shown in FIG. 5 is the cord sling device 100 being utilized to retain a plurality of cords and cables 350 while being hung from an IV pole 300. The cord sling device 100 provides users with a safer, more organized, and visually appealing environment for cords and cables 350. Further, the device protects cords and cables 350 from wear and tear and protects medical equipment from overheating issues by providing better airflow. The device 100 can also be easily cleaned between patients and reused if desired.

It is to be appreciated that the cord sling device 100 in accordance with the present invention can be produced in various colors, designs, patterns, etc., and feature logos, emblems and or designs, such as a medical facility requests and/or a user 108 desires.

In one embodiment, the cord sling device 100 is manufactured of a high-density polyethylene (HDPE), or any other suitable materials as is known in the art. Any number of different types of materials can be used to make the cord sling device 100 including but not limited to nylon, heat-sealable plastic or polymers, such as polypropylene or acrylonitrile-butadiene-styrene (ABS), or any other suitable material as is known in the art, such as but not limited to, acrylic, polycarbonate, polyethylene, polyethylene terephthalate, polyvinyl chloride, polystyrene, etc. Generally, the cord sling device 100 is also manufactured from a material that is water resistant or waterproof, or the body component 110 comprises a coating that is water resistant or waterproof.

In yet another embodiment, the cord sling device 100 comprises a plurality of indicia 400. The body component 110 of the device 100 may include advertising, a trademark, or other letters, designs, or characters, printed, painted, stamped, or integrated into the body component 110, or any other indicia 400 as is known in the art. Specifically, any suitable indicia 400 as is known in the art can be included, such as but not limited to, patterns, logos, emblems, images, symbols, designs, letters, words, characters, animals, advertisements, brands, etc., that may or may not be healthcare or brand related.

It will also be appreciated that there are a number of additional add-on features that can be incorporated into the device 100 and moreover, the cord sling device 100 can take many different forms as is known in the art.

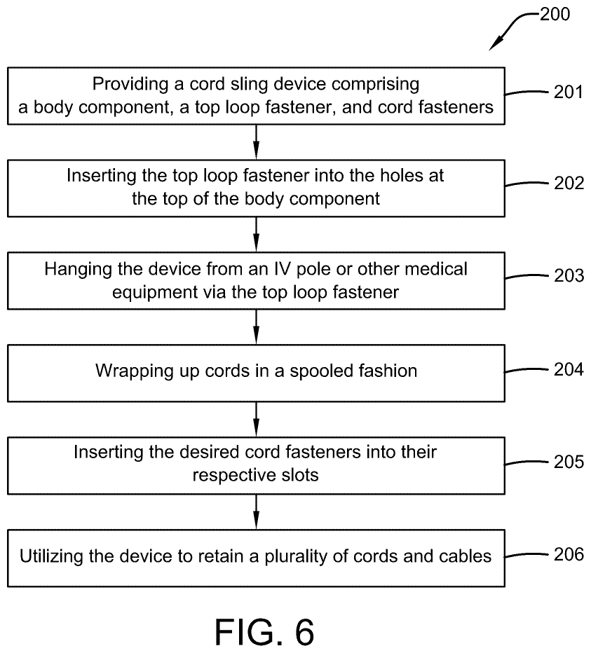

FIG. 6 illustrates a flowchart of the method 200 of conveniently retaining a plurality of cords and cables. The method includes the steps of at 201, providing a cord sling device comprising a body component, a top loop fastener, and cord fasteners. The method also comprises at 202, inserting the top loop fastener into the holes at the top of the body component. Further, the method comprises at 203, hanging the device from an IV pole or other medical equipment via the top loop fastener. Next, the method comprises at 204, wrapping up cords in a neatly spooled fashion. Additionally, the method comprises at 205, inserting the desired cord fasteners into their respective slots. Finally, the method comprises at 206, utilizing the device to retain a plurality of cords and cables.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “cord sling device”, “sling device”, “cord device”, and “device” are interchangeable and refer to the cord sling device 100 of the present invention.

Notwithstanding the forgoing, the cord sling device 100 of the present invention can be of any suitable size and configuration as is known in the art without affecting the overall concept of the invention, provided that it accomplishes the above stated objectives. One of ordinary skill in the art will appreciate that the cord sling device 100 as shown in FIGS. 1-5 is for illustrative purposes only, and that many other sizes and shapes of the cord sling device 100 are well within the scope of the present disclosure. Although the dimensions of the cord sling device 100 are important design parameters for user convenience, the cord sling device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A cord sling device comprising:
a body component configured to be attached to a pole and having a plurality of holes, a plurality of slots, a top loop fastener, and
a plurality of cord fasteners;
wherein said body component secured proximal to a top of said pole with said top loop fastener wrapped around an arm of said pole and inserted into one of said plurality of holes;
wherein each of said plurality of cord fasteners having a flexible male end for inserting into a selected one of said plurality of slots; and
further wherein at least one of said plurality of cord fasteners fastened around a portion of a spooled cord and inserted into one of said plurality of slots for suspending the spooled cord above the ground.

2. The cord sling device of claim 1, wherein said pole is an IV pole.

3. The cord sling device of claim 2, wherein said body component having a pair of said plurality of cord fasteners for suspending a pair of spooled cords above the ground.

4. The cord sling device of claim 2, wherein said top loop fastener having a flexible T-shaped male end.

5. The cord sling device of claim 4, wherein said plurality of cord fasteners are rectilinear having a plurality of ridges along opposing sides of said plurality of cord fasteners.

6. The cord sling device of claim 5, wherein said body component having a material of a high-density polyethylene (HDPE).

7. The cord sling device of claim 5, wherein said body component having a material selected from the group consisting of a nylon, a heat-sealable polymer, a polypropylene, and an acrylonitrile-butadiene-styrene (ABS).

8. The cord sling device of claim 5, wherein said body component having a material selected from the group consisting of an acrylic, a polycarbonate, a polyethylene, a polyethylene terephthalate, a polyvinyl chloride, and a polystyrene.

9. The cord sling device of claim 8, wherein said body component having a waterproof material.

10. A cord sling device comprising:
a body component having a plurality of holes, a plurality of slots, a top loop fastener, and a plurality of cord fasteners;
an IV pole;
wherein said body component secured proximal to a top of said IV pole with said top loop fastener wrapped around an arm of said IV pole and inserted into one of said plurality of holes;
wherein each of said plurality of cord fasteners having a flexible male end for inserting into a selected one of said plurality of slots;
wherein at least one of said plurality of cord fasteners fastened around a portion of a spooled cord and inserted into one of said plurality of slots for suspending the spooled cord above the ground; and
further wherein said top loop fastener having a flexible T-shaped male end.

11. The cord sling device of claim 10, wherein said body component having a pair of said plurality of cord fasteners for suspending a pair of spooled cords above the ground.

12. The cord sling device of claim 10, wherein said plurality of cord fasteners are rectilinear having a plurality of ridges along opposing sides of said plurality of cord fasteners.

13. The cord sling device of claim 12, wherein said body component having a material of a high-density polyethylene (HDPE).

14. The cord sling device of claim 12, wherein said body component having a material selected from the group consisting of a nylon, a heat-sealable polymer, a polypropylene, and an acrylonitrile-butadiene-styrene (ABS).

15. The cord sling device of claim 12, wherein said body component having a material selected from the group consisting of an acrylic, a polycarbonate, a polyethylene, a polyethylene terephthalate, a polyvinyl chloride, and a polystyrene.

16. The cord sling device of claim 15, wherein said body component having a waterproof material.

17. A method of using a cord sling device for an IV pole, the method comprising the steps of:
providing a body component having a plurality of holes, a plurality of slots, a top loop fastener, a plurality of cord fasteners, and an IV pole, wherein said top loop fastener having a flexible T-shaped male end;
securing said body component proximal to a top of said IV pole with said top loop fastener wrapped around an arm of said IV pole and inserted into one of said plurality of holes, wherein each of said plurality of cord fasteners having a flexible male end for inserting into a selected one of said plurality of slots;
wrapping at least one of said plurality of cord fasteners around a portion of a spooled cord; and
inserting a male end of said at least one of said plurality of cord fasteners into one of said plurality of slots for suspending the spooled cord above the ground.

18. The method of using a cord sling device for an IV pole of claim 17, wherein said body component having a pair of said plurality of cord fasteners for suspending a pair of spooled cords above the ground.

19. The method of using a cord sling device for an IV pole of claim 17, wherein said plurality of cord fasteners are rectilinear having a plurality of ridges along opposing sides of said plurality of cord fasteners.

20. The method of using a cord sling device for an IV pole of claim 17, wherein said body component having a material selected from the group consisting of a nylon, a heat-sealable polymer, a polypropylene, and an acrylonitrile-butadiene-styrene (ABS), and further wherein said body component having a waterproof material.
